SEQUENCE LISTING (19 sequences)

SEQ ID NO: 1 - protein, 15 aa, Lactobacillus reuteri
MKNIMIAGAGVLGSQ

SEQ ID NO: 2 - protein, 12 aa, Lactobacillus reuteri
  Peptide (1)..(12)
SDYERDLHLTDK

SEQ ID NO: 3 - DNA, 26 bp, Artificial sequence
  designed primer for Seq. No. 1
atgaaraayatyatgatygchggcgc

SEQ ID NO: 4 - DNA, 26 bp, Artificial Sequence
  designed primer for Seq. No. 1
atgaaraayatyatgatygchggtgc

SEQ ID NO: 5 - DNA, 21 bp, Artificial Sequence
  designed primer for Seq. No. 2
rtgharatcmcgttcrtaatc

SEQ ID NO: 6 - DNA, 21 bp, Artificial Sequence
  designed primer for Seq. No. 2
rtgharatcmcgttcrtagtc

SEQ ID NO: 7 - DNA, 159 bp, Lactobacillus reuteri
atgaagaatatcatgattgctggtgctggcgtattaggtagtcagattgcatatcaaacggctttatccggctttaatgtcagcgtatataatcaccatattgataccgctgagcgacggattaaggcgctgaaaagtgattacgaacgtgatttacat

SEQ ID NO: 8 - DNA, 21 bp, Artificial Sequence
  designed primer to 3′ end of Seq. No. 7
atccggctttaatgtcagcgt

SEQ ID NO: 9 - DNA, 23 bp, Artificial Sequence
  designed primer to 3′ end of Seq. No. 7
cgacggattaaggcgctgaaaag

SEQ ID NO: 10 - DNA, 22 bp, Artificial Sequence
  designed primer to 5′ end of Seq. No. 7
ctacctaatacgccagcaccag

SEQ ID NO: 11 - DNA, 18 bp, Artificial Sequence
  designed primer to 5′ end of Seq. No. 7
gccagcaccagcaatcat

SEQ ID NO: 12 - DNA, 1257 bp, Lactobacillus reuteri
cgacggattaaggcgctgaaaagtgattatgaacgtgacttgcatttgactgataaggaatttcaacagggccttaataatattaaagtgattactgatgatgttgcgaccgcggttaaagatgctgatttaatgattgaagcattaccagaatcgttagagttaaaggagcagttttacgaagaggtttcagaattagcccctgaaaaaacaatctttgctagcaactcctctacatttatccctagccaactagctccttatactgatcggccagaaaaatttcttaatatgcactttgctaaccaaatttggaaatttaatgtggtcgaaattatgggcacctcccaaacaagtccagaggtgattgaagaagctacaaagtttgcccgggaaataaagatggttcctgttatccttaataaagaacaacacggttatattctgaattcatggtgaatcaggaatgcctggttatgactttggtgacgattatggtaagcctcttgataagactgctgttgaagcacttcatgacgcaatcatggctgaagatgaagtcatcttggttccaactggttcatacactaacattgcattactctttagcgaatacccagaagtaaagagtcacattaagcaaatcgttgcgatgggtggttcattctctggcggtaacatgaccagtgtagctgaatttaacgtcttcactgatccggatgctgctaagattatgtacaatgcgggtgttccaattgtaactgttggattggatgttaccttaaaggcgctcttaactgcggatacgattgaaaaacttggtagtcttaataagactggtgaaatgctacatggattaatcacgcactataatgatggtagtgatcaagggcgtccaatgcacgatgttaatactatcttctaccttcttcatccagaagcatttactacgaaggacatgtgggttgatgttcaaacagacggtccagcaatcggtgctactgttggtgacattcgcgctgcttaccatgatggcaagaccaacgcaaaagtttgtttagatattgatgctgaatacttcaataagtggttcttagaagaagtaagcaaaatgaaataagatactaagagctgagagtttgctttcggctctttttattactttctagtgtaagcgcttcacgatataatagtaatagttaaagtaaggaggaaataccaaatgaagaatataatgattgctggtgctggcaagcc

SEQ ID NO: 13 - DNA, 23 bp, Artificial Sequence
  designed primer to 3′ end of Seq. No. 12
ccagaggtgattgaagaagctac

SEQ ID NO: 14 - DNA, 19 bp, Artificial Sequence
  designed primer to 3′ end of Seq. No. 12
ccgggaaataaagatggtt

SEQ ID NO: 15 - DNA, 822 bp, Lactobacillus reuteri
ccgggaaataaagatggttcctgttatccttaataaagaacaacacggttatattctgaattcccttttgattccgttacttgcatctggtcttagtttatgggctaaaggagttgctgatccagcgatgattgataaggattggatgatttcgacaggtgcaccaatgggaccatttggtattttagatatggttggtttacggacagcagcgcaaatcgaacggaatgcctatgcccagactaaggatgaaagtcataaggaaattgccgataagatggaacagatgattcaagaagggcacgaaggaaaagaatcgggacaaggattttataattatccgaatccagccttcatggatccagattttctgaagcattaagattaagtattgaaaacgttaggaaagggagcaagataagagtcgctcgtgactgttatctcactcccttattttgcgctccaattttcacgtaatccggttgcaacaatggcaaaattacggtacagtagtagagaacgtgaaacaaaaaatgtttcacgaaaggagacctaataaatgaatcctgaacaatttcaacaagcattagctgatcatggtattaccttatcagcagaacaaatgcaacaatttgctgactattaccaattattagttgaaactaatgaacatggtgggttgatgttcaacagacggtccagcatcggtgctactgtggtgacattcgcgctgcttacatgatggcagaccaacgcaaagtttgttagatatgatgctgaatacttcaataagtggttcttaaagaagtagcaaatgaatag

SEQ ID NO: 16 - DNA, 33 bp, Artificial Sequence
  designed 5′-Primer of recognition sequence for Nde I
gcggaattccatatgaagaatatcatgattgct

SEQ ID NO: 17 - DNA, 28 bp, Artificial Sequence
  designed 3′-Primer of recognition sequence for Hind III
cccaagcttaatgcttcagaaaatctgg

SEQ ID NO: 18 - protein, 294 aa, Lactobacillus reuteri
MKNIMIAGAGVLGSQIAYQTALSGFNVSVYNHHIDTAERRIKALKSDYERDLHLTDKEFQQGLNNIKVITDDVATAVKDADLMIEALPESLELKEQFYEEVSELAPEKTIFASNSSTFIPSQLAPYTDRPEKFLNMHFANQIWKFNVVEIMGTSQTSPEVIEEATKFAREIKMVPVILNKEQHGYILNSLLIPLLASGLSLWAKGVADPAMIDKDWMISTGAPMGPFGILDMVGLRTAAQIERNAYAQTKDESHKEIADKMEQMIQEGHEGKESGQGFYNYPNPAFMDPDFLKH

SEQ ID NO: 19 - DNA, 885 bp, Lactobacillus reuteri
atgaagaatatcatgattgctggtgctggcgtattaggtagtcagattgcatatcaaacggctttatccggctttaatgtcagcgtatataatcaccatattgataccgctgagcgacggattaaggcgctgaaaagtgattatgaacgtgacttgcatttgactgataaggaatttcaacagggccttaataatattaaagtgattactgatgatgttgcgaccgcggttaaagatgctgatttaatgattgaagcattaccagaatcgttagagttaaaggagcagttttacgaagaggtttcagaattagcccctgaaaaaacaatctttgctagcaactcctctacatttatccctagccaactagctccttatactgatcggccagaaaaatttcttaatatgcactttgctaaccaaatttggaaatttaatgtggtcgaaattatgggcacctcccaaacaagtccagaggtgattgaagaagctacaaagtttgcccgggaaataaagatggttcctgttatccttaataaagaacaacacggttatattctgaattcccttttgattccgttacttgcatctggtcttagtttatgggctaaaggagttgctgatccagcgatgattgataaggattggatgatttcgacaggtgcaccaatgggaccatttggtattttagatatggttggtttacggacagcagcgcaaatcgaacggaatgcctatgcccagactaaggatgaaagtcataaggaaattgccgataagatggaacagatgattcaagaagggcacgaaggaaaagaatcgggacaaggattttataattatccgaatccagccttcatggatccagattttctgaagcattaa

(19) United States
(12) Patent Application Publication
(10) Pub. No.: US 2005/0191735 A1
(43) Pub. Date: Sep. 1, 2005

(54) Oxidoreductase

(76) Inventors: Maria Bobkova, Idstein (DE); Antje Gupta, Wiesbaden (DE); Anke Zimmer, Wiesbaden (DE)

Correspondence Address: PROPAT, L.L.C., 425-C SOUTH SHARON AMITY ROAD, CHARLOTTE, NC, 28211-2841, US

(21) Appl. No.: 10/753,022

(22) Filed: Jan. 7, 2004

(30) Foreign Application Priority Data
Jan. 9, 2003 (DE) 103 00 335.5.0

(51) Int. Cl. C12P 7/62; C07H 21/04; C12N 9/02; C12N 1/21; C12N 15/74

(52) U.S. Cl. 435/135; 435/69.1; 435/189; 435/320.1; 435/252.3; 536/23.2

(57) ABSTRACT

The present invention relates to an NADPH-dependent oxidoreductase available from Lactobacilli, to an enzymic method for enantioselective reduction of 2-oxo acid esters to the corresponding chiral S-2-hydroxy acid esters and to an enzymic method of enantioselectively obtaining S-2-hydroxy acid esters by enzyme-coupled coenzyme regeneration.

### FIELD OF THE INVENTION

The present invention relates to an oxidoreductase, to a fragment and an isolated DNA sequence of said oxidoreductase, to a fusion protein based on said oxidoreductase or said fragment and to a method for enantioselectively obtaining S-2-hydroxy acid esters.

### BACKGROUND OF THE INVENTION

2-Hydroxy acids and esters thereof are important chiral basic synthetic building blocks from which a number of compounds can be derived while preserving the chirality at the C2 atom, for example epoxides, alkyl esters, hydrazinyl esters, alpha-N-alkoxyamino esters or alpha-amino esters.

Numerous research studies have been devoted previously to the development of methods for preparing enantiomerically pure 2-hydroxy acids and esters thereof, various chemical and biocatalytic approaches having been contemplated. Up until now it has not been possible to develop methods which meet the demands of production on the industrial scale. The enzyme-catalyzed introduction of the chiral center appears to be superior to synthesis via chemical catalysts, in particular in the preparation of 2-hydroxy acids and their esters.

The enzyme-catalyzed methods currently comprise three different methods. One route is the oxynitrilase-catalyzed synthesis of chiral cyanohydrins and subsequent hydrolysis thereof which is frequently also enzyme-catalyzed (Biotransformations in Organic Chemistry, A Textbook. 4th edition, Springer (2000), K. Faber; Cyanohydrin formation). This method has the disadvantage of using the toxic HCN.

Another method is the resolution of 2-hydroxy acid esters with the aid of lipases, for example from Pseudomonas fluorescens (J. Org. Chem. 55, 812-815 (1991), Kinetic Resolution of 2-substituted Esters catalysed by a Lipase Ex. Pseudomonas fluoreszens, Kalaritis, P. et al.). The disadvantage of this method is the theoretical yield of only 50%.

Another method is the synthesis of chiral 2-hydroxy acids and esters thereof by reducing prochiral 2-oxo acids or esters thereof. Transformation with whole yeast cells or with cells of Proteus vulgaris or Proteus mirabilis or methods using isolated enzymes is known. In the case of transformations with whole yeast cells, the reducing enzyme action on 2-oxo acids was attributed to the enzymes lactate dehydrogenase or malate dehydrogenase (Ramesh N. Patel Stereoselective Biocatalyse, NY (2000), 14. Stereoselective Synthesis of Chiral Compounds Using Whole-Cell Biocatalysis, Paola D'Arrigo, Giuseppe Pedrocchi-Fantoni and Stefano Servi), while, in the reductions carried out with Proteus, a membrane-bound molybdenum-dependent iron-sulfur protein is apparently responsible for the reaction (Eur J Biochem (1994); 222 (3): 1025-32, The (2R)-hydroxycarboxylate-viologen-oxidoreductase from Proteus vulgaris is a molybdenum-containing iron-sulphur protein, Trautwein T, Krauss F, Lottspeich F, Simon H).

In known methods for reducing 2-oxo acid esters, the isolated enzymes (D/L)-lactate dehydrogenase (U.S. Pat. No. 5,686,275), (D/L)-dihydroxyisocaproate dehydrogenase (U.S. Pat. No. 6,033,882) and D-mandelate dehydrogenase (Appl Environ Microbiol 2002 February; 68 (2): 947-51, Two forms of NAD-dependent D-mandelate dehydrogenase in Enterococcus faecalis IAM 10071. Tamura Y. et al 10) are used, which are also available in cloned, overexpressed and commercial form. These enzymes are NADH-dependent and do not convert 2-oxo acid esters. It is furthermore known that enzymes reducing 2-oxo acids or esters thereof do not convert secondary alcohols.

Furthermore, methods are known which comprise regenerating the coenzyme NAD with formate dehydrogenase, for example from Candida boidinii or else in recombinant form from Pseudomonas fluoreszens (Biotechnology, Biotransformations I (Rehm and Reed) 9. Alcohol Dehydrogenases-Characteristics, Design of Reaction Conditions J. Peters, WILEY-VCH-Verlag, (1998)). The method of enzymatically preparing R-2-hydroxy-4-phenylbutyric acid with the aid of D-lactate dehydrogenase from Staphylococcus epidermidis is mentioned here by way of example (Industrial Biotransformations, Liese, K. Seelbach, C. Wandrey, WILEY-VCH-Verlag, (2000)). A disadvantage of coenzyme regeneration using formate dehydrogenase is the low specific activity of said formate dehydrogenase (4 to 10 U/mg) and the high costs of preparing the enzyme. From an economic viewpoint it is therefore necessary to use the enzyme several times, resulting in a comparatively substantially more complex and thus more expensive process control.

### SUMMARY OF THE INVENTION

It is the object of the invention to remove the disadvantages mentioned of the methods of the prior art by means of an oxidoreductase.

According to the invention, this object is achieved by an oxidoreductase reducing 2-oxo acid esters to the corresponding S-2-hydroxy acid esters in the presence of NADPH and water.

### DETAILED DESCRIPTION OF THE INVENTION

The invention relates, inter alia, to oxidoreductases which may be obtained from Lactobacillus (L.) reuteri, L. kefiri, L. kandleri, L. parabuchneri, L. cellobiosus or L. fermentum, for example.

The invention further relates to the Lactobacillus reuteri oxidoreductase which has the DNA sequence according to SEQ ID NO: 19 and the amino acid sequence according to SEQ ID NO: 18 as described in the attached sequence listing.

The invention further relates to an oxidoreductase wherein more than 70% of the amino acids therein are identical to the amino acid sequence SEQ ID NO: 18 and which has a specific activity of more than 1 mmol per mg, based on the conversion of ethyl 2-oxo-4-phenylbutyrate to ethyl S-2-hydroxy-4-phenylbutyrate. Preference is given to oxidoreductases wherein from 80% to 99.5%, in particular from 90% to 99.5%, particularly preferably from 99% to 99.5%, of the amino acids are identical to the amino acid sequence of SEQ ID NO: 18. The specific activity of the oxidoreductase according to SEQ ID NO: 18 or of its derivatives or analogs is measured using the assay system described in example 2.

The invention further relates to an oxidoreductase which has from 1 to 50 amino acids more or from 1 to 50 amino acids fewer than the oxidoreductase having the amino acid sequence SEQ ID NO: 18 and a specific activity of more than 1 μmol per mg, based on the conversion of ethyl 2-oxo-4-phenylbutyrate to ethyl S-2-hydroxy-4-phenylbutyrate. Preference is given to oxidoreductases which have from 1 to 25 amino acids, in particular from 2 to 20 amino acids, preferably from 3 to 10 amino acids, more or fewer than occur in the amino acid sequence of SEQ ID NO: 18.

The invention further relates to an oxidoreductase which has the amino acid sequence of SEQ ID NO: 18 and has been modified once, twice, three, four or five times by a water-soluble polymer and has a specific activity of more than 1 mmol per mg, based on the conversion of ethyl 2-oxo-4-phenylbutyrate to ethyl S-2-hydroxy-4-phenylbutyrate. An example of a water-soluble polymer is polyethylene glycol. Polyethylene glycol is preferably bound to the N-terminal end of the oxidoreductase according to SEQ ID NO: 18. The oxidoreductase according to SEQ ID NO: 18 may also be bound to a solid such as polyethylene, polystyrene, polysaccharide, cellulose or cellulose derivative.

The invention further relates to an oxidoreductase fragment which represents a fragment of the amino acid sequence SEQ ID NO: 18, having from 5 to 30 amino acids. Preference is given to a fragment of SEQ ID NO: 18 which has an amino acid chain of from 6 to 25 amino acids, in particular from 8 to 20 amino acids, preferably from 10 to 18 amino acids, in particular the amino acid sequences SEQ ID NO: 1 and SEQ ID NO: 2. Fragments of this kind may be used, for example, for finding the inventive oxidoreductase from L. reuteri or from any other microorganisms.

The invention further relates to a fusion protein which represents the oxidoreductase having the amino acid sequence SEQ ID NO: 18 or a fragment thereof having from 5 to 30 amino acids and said oxidoreductase or said fragment thereof being linked at the N terminus or carboxy terminus via a peptide bond to another polypeptide. Fusion proteins can be removed relatively easily from other proteins, for example, or are expressed in relatively large quantities in the cells.

The invention further relates to an antibody which binds specifically to the oxidoreductase according to SEQ ID NO: 18 or to a fragment thereof according to SEQ ID NO: 1 or SEQ ID NO: 2. These antibodies are prepared according to known methods by immunizing suitable mammals such as horse, mouse, rat or pig and subsequently obtaining said antibodies. The antibodies may be monoclonal or polyclonal.

The invention also relates to an isolated nucleic acid sequence which codes for the oxidoreductases according to SEQ ID NO: 18, SEQ ID NO: 1 or SEQ ID NO: 2.

The invention further relates to an isolated deoxyribonucleic acid sequence (DNA sequence) of the oxidoreductase catalyzing the reduction of 2-oxo acid esters to corresponding S-2-hydroxy acid esters in the presence of NADPH and water, wherein said DNA sequence is selected from the group consisting of
a) a DNA sequence having the nucleotide sequence according to SEQ ID NO: 7, SEQ ID NO: 12, SEQ ID NO: 15 or SEQ ID NO: 19 or the in each case complementary strands,
b) a DNA sequence hybridizing to one or more of the DNA sequences according to a) or to their complementary strands, said hybridization being carried out under stringent conditions, and
c) a DNA sequence encoding, owing to the degeneracy of the genetic code, a protein which is encoded by one or more of the DNA sequences according to a) or b).

Hybridization is described, for example, by Sambrook and Russel in Molecular Cloning a laboratory Manual, volume 1, chapter 1, protocol 30-32.

The invention further relates to an isolated DNA sequence wherein more than 70% of the nucleic acid bases are identical to the DNA sequence according to SEQ ID NO: 7, SEQ ID NO: 12, SEQ ID NO: 15 or SEQ ID NO: 19 or to the complementary strands thereof and which encodes a protein having a specific activity of more than 1 μmol per mg, based on the conversion of ethyl 2-oxo-4-phenylbutyrate to ethyl S-2-hydroxy-4-phenylbutyrate. Preference is given to DNA sequences wherein from 80% to 99.5%, in particular from 90% to 99.5%, preferably from 99% to 99.5%, of the nucleic acid bases are identical to the DNA sequence according to SEQ ID NO: 7, SEQ ID NO: 12, SEQ ID NO: 15 or SEQ ID NO: 19.

The invention further relates to an isolated DNA sequence having from 10 to 50 nucleic acid bases and a sequence corresponding to part of a DNA sequence according to SEQ ID NO: 7, SEQ ID NO: 12, SEQ ID NO: 15 or SEQ ID NO: 19 or to the complementary strand thereof. Preference is given to a nucleic acid sequence having from 15 to 45 nucleic acid bases, in particular from 20 to 40 bases, particularly preferably from 30 to 40 nucleic acid bases. The nucleic acid sequences mentioned are suitable as molecular samples or as primers for the polymerase chain reaction (PCR).

The invention further relates to a cloning vector comprising one or more of the nucleic acid or DNA sequences mentioned above. The invention further relates to an expression vector which is present in a bacterial, yeast, insect, plant or mammalian cell and which comprises one or more of the nucleic acid or DNA sequences mentioned above and which is linked in a suitable manner to an expression control sequence.

The invention further relates to a host cell which is a bacterial, yeast, insect, plant or mammalian cell and which has been transformed or transfected with any of the abovementioned expression vectors.

The homologies of the abovementioned DNA sequences or amino acid sequence are calculated by adding up the number of amino acids or nucleic acid bases identical to partial sequences of the respective proteins or DNA sequences, dividing this by the total number of amino acids or nucleic acid bases and multiplying by one hundred.

Examples of suitable cloning vectors are ppCR-Script, pCMV-Script, pBluescript (Stratagene), pDrive cloning Vector (Quiagen), pS Blue, pET Blue, pET LIC vectors (Novagen) and TA-PCR cloning vectors (Invitrogen).

Examples of suitable expression vectors are pKK223-3, pTrc99a, pUC, pTZ, pSK, pBluescript, pGEM, pQE, pET, PHUB, pPLc, pKC30, pRMl/pRM9, pTrxFus, pAS1, pGEx, PMAL, pTrx).

Examples of suitable expression control sequences are trp-lac (tac) promoter, trp-lac (trc) promoter, lac promoter, T7 promoter, XpL promoter.

The Lactobacillus reuteri oxidoreductase is a homodimer having a molecular weight of from 30 to 35 kDa, as determined in an SDS gel, and a molecular weight of from 60 to 65 kDa, as determined by gel permeation chromatography. The optimal temperature is in the range from 55° C. to 60° C. and its optimal pH is from 6.5 to 7.0. Lactobacillus reuteri oxidoreductase has good temperature and pH stabilities and is stable for at least 5 hours within a pH range from 4.5 to 8.5 and a temperature range from 15° C. to 50° C. and furthermore exhibits high stability in organic solvents.

The enzyme can be isolated in particular from microorganisms of the genus Lactobacillus and detected in a spectrophotometric assay via the decrease in NADPH at 340 nm in the presence of an appropriate substrate, for example ethyl 2-oxo-4-phenylbutyric acid or ethyl 2-oxovaleric acid.

The Lactobacillus reuteri oxidoreductase of the invention was cloned and overexpressed in Escherichia (E.) coli, with activities of from 10 000 U/g to 30 000 U/g of E. coli wet weight. The enzyme is thus inexpensive and available in large quantities. No related sequences were found in databases, and only a distant relationship to enzymes of the group of hydroxyacyl-CoA dehydrogenases might be suspected. The invention also relates to a method for obtaining Lactobacillus reuteri oxidoreductase. For this purpose, the DNA coding for Lactobacillus reuteri oxidoreductase is expressed in a suitable prokaryotic or eukaryotic microorganism. Preference is given to Lactobacillus reuteri oxidoreductase being transformed into and expressed in an Escherichia coli strain, in particular Escherichia coli BL21star (DE3) cells (Invitrogen, cat. No. C6010-03, derived from E. coli BL21, with a chromosomal copy of the T7 RNA polymerase gene under the control of the lacUV5 promoter, without ompT and Lon protease, B121 star has mutation in RNaseE (rnel31).

Lactobacillus reuteri oxidoreductase can be obtained, for example, by culturing the recombinant Escherichia coli cells, inducing expression of said oxidoreductase and subsequently, after approximately 10 to 18 hours (h), disrupting said cells by ultrasound treatment or by wet grinding with glass beads in a ball mill (Retsch, 10 min, 24 Hz). The cell extract obtained may either be used directly or be purified further. For this purpose, the cell extract is centrifuged, for example, and the supernatant obtained is subjected to hydrophobic interaction chromatography, for example hydrophobic interaction chromatography on Butyl Sepharose Fast Flow (Pharmacia) and subsequent gel permeation (Superdex 200 HR, Pharmacia).

The invention further relates to a method for enantioselectively obtaining S-2-hydroxy acid esters, which comprises reducing 2-oxo acid esters in the presence of oxidoreductase, NADPH and water to the corresponding S-2-hydroxy acid ester and isolating the S-2-hydroxy acid ester produced.

The method of the invention has a long useful life, an enantiomeric purity of more than 94% of the chiral S-2-hydroxy acid esters prepared and a high yield based on the amount used of the 2-oxo acid ester.

The term “NADPH” means reduced nicotinamide adenine dinucleotide phosphate. The term “NADP” means nicotinamide adenine dinucleotide phosphate.

The term “2-oxo acid esters” means, for example, compounds of the formula I
R2—C(O)—C(O)—O—R1 (I).
R1 is
1. —(C₁-C₂₀)-alkyl where alkyl is straight-chain or branched,
2. —(C₂-C₂₀)-alkenyl where alkenyl is straight-chain or branched and comprises one, two, three or four double bonds, depending on the chain length,
3. —(C₂-C₂₀)-alkynyl where alkynyl is straight-chain or branched and comprises one, two, three or four triple bonds, where appropriate,
4. —(C₆-C₁₄)-aryl,
5. —(C₁-C₈)-alkyl-(C₆-C₁₄)-aryl,
6. —(C₅-C₁₄)-heterocycle which is unsubstituted or mono- to trisubstituted by halogen, hydroxyl, amino or nitro, or
7. —(C₃-C₇)-cycloalkyl,
   R2 is
1.—(C₁-C₂₀)-alkyl where alkyl is straight-chain or branched,
2. —(C₂-C₂₀)-alkenyl where alkenyl is straight-chain or branched and comprises one, two, three or four double bonds, depending on the chain length,
3. —(C₂-C₂₀)-alkynyl where alkynyl is straight-chain or branched and comprises one, two, three or four triple bonds, where appropriate,
4. —(C₆-C₁₄)-aryl,
5. —(C₁-C₈)-alkyl-(C₆-C₁₄)-aryl,
6. —(C₅-C₁₄)-heterocycle which is unsubstituted or mono- to trisubstituted by halogen, hydroxyl, amino or nitro, or
7. —(C₃-C₇)-cycloalkyl, wherein the radicals as defined above under 1. to 7. are unsubstituted or, independently of one another, mono- to trisubstituted by
   a) —OH,
   b) halogen such as fluorine, chlorine, bromine or iodine,
   c) —NO₂,
   d) —C(O)—O—(C₁-C₂₀)-alkyl where alkyl is linear or branched and unsubstituted or mono- to trisubstituted by halogen, hydroxyl, amino or nitro, or
   e) —(C₅-C₁₄)-heterocycle which is unsubstituted or mono- to trisubstituted by halogen, hydroxyl, amino or nitro.

The term “S-2-hydroxy acid esters” means compounds of the formula II
R2—C(OH)—C(O)—O—R1 (II)
where the —OH group is in S configuration with respect to the carbon atom to which it is bound and R1 and R2 are as defined in formula I.

The term aryl means aromatic carbon radicals having from 6 to 14 ring carbons. Examples of —(C₆-C₁₄)-aryl radicals are phenyl, naphthyl, for example 1-naphthyl, 2-naphthyl, biphenylyl, for example 2-biphenylyl, 3-biphenylyl and 4-biphenylyl, anthryl or fluorenyl. Preferred aryl radicals are biphenylyl radicals, naphthyl radicals and in particular phenyl radicals. The term “halogen” means an element of the series fluorine, chlorine, bromine and iodine. The term “—(C₁-C₂₀)-alkyl” means a hydrocarbon radical whose carbon chain is straight-chain or branched and comprises from 1 to 20 carbon atoms, for example methyl, ethyl, propyl, isopropyl, butyl, tert-butyl, pentyl, hexyl, heptyl, octyl, nonenyl or decanyl.

The term “—(C₃-C₇)-cycloalkyl” means cyclic hydrocarbon radicals such as cyclopropyl, cyclobutyl, cyclopentyl, cyclohexyl or cycloheptyl.

The term “—(C₅-C₁₄)-heterocycle” is a monocyclic or bicyclic 5-membered to 14-membered heterocyclic ring which is partially or completely saturated. Examples of heteroatoms are N, O and S. Examples of the terms —(C₅-C₁₄)-heterocycle are radicals deriving from pyrrole, furan, thiophene, imidazole, pyrazole, oxazole, isoxazole, thiazole, isothiazole, tetrazole, 1,2,3,5-oxathiadiazole-2-oxides, triazolones, oxadiazolones, isoxazolones, oxadiazolidindiones, triazoles, substituted by F, —CN, —CF3 or —C(O)—O—(C₁-C₄)-alkyl, 3-hydroxypyrro-2,4-diones, 5-oxo-1,2,4-thiadiazoles, pyridine, pyrazine, pyrimidine, indole, isoindole, indazole, phthalazine, quinoline, isoquinoline, quinoxaline, quinazoline, quinnoline, -carboline and benzo-, cyclopenta-, cyclohexa- or cyclohepta-fused derivatives of said heterocycles. Particular preference is given to the radicals 2- or 3-pyrrolyl, phenylpyrrolyl such as 4- or 5-phenyl-2-pyrrolyl, 2-furyl, 2-thienyl, 4-imidazolyl, methylimidazolyl, for example 1-methyl-2-, -4- or -5-imidazolyl, 1,3-thiazol-2-yl, 2-pyridyl, 3-pyridyl, 4-pyridyl, 2-, 3- or 4-pyridyl-N-oxide, 2-pyrazinyl, 2-, 4- or 5-pyrimidinyl, 2-, 3- or 5-indolyl, substituted 2-indolyl, for example 1-methyl-, 5-methyl-, 5-methoxy-, 5-benzyloxy-, 5-chloro- or 4,5-dimethyl-2-indolyl, 1-benzyl-2- or -3-indolyl, 4,5,6,7-tetrahydro-2-indolyl, cyclohepta[b]-5-pyrrolyl, 2-, 3- or 4-quinolyl, 1-, 3- or 4-isoquinolyl, 1-oxo-1,2-dihydro-3-isoquinolyl, 2-quinoxalinyl, 2-benzofuranyl, 2-benzothienyl, 2-benzoxazolyl or benzothiazolyl or dihydropyridinyl, pyrrolidinyl, for example 2- or 3-(N-methylpyrrolidinyl), piperazinyl, morpholinyl, thiomorpholinyl, tetrahydrothienyl or benzodioxolanyl.

Examples of preferred compounds of the formula I are ethyl 2-oxovalerate, ethyl 2-oxo-4-phenylbutyrate, ethyl pyruvate, ethyl phenylglyoxylate, ethyl 2-oxo-3-phenylpropionic acid, ethyl 8-chloro-6-oxooctanoate, ethyl 2-oxobutyrate, ethyl 2-oxohexanoate, methyl phenylglyoxylate, methyl 2-oxovalerate, methylpyruvate, methyl 2-oxo-4-phenylbutyrate, methyl 2-oxo-3-phenylpropionic acid, methyl 8-chloro-6-oxooctanoate, methyl 2-oxobutyrate and methyl 2-oxohexanoate.

The correspondingly produced S-2-hydroxy acid esters are, for example, ethyl S-2-hydroxyvalerate, ethyl S-2-hydroxy-4-phenylbutyrate, ethyl L-lactate or ethyl S-mandelate.

Suitable oxidoreductases are derived from Lactobacillus reuteri, for example. It is possible to use in the method of the invention either a completely or partially purified oxidoreductase or an oxidoreductase contained in cells. The cells used in this connection may be in native, permeabilized or lysed form. Preference is given to using the cloned oxidoreductase according to SEQ ID NO: 18.

The volume activity of the oxidoreductase used is from 250 units/ml (U/ml) to 20 000 U/ml, preferably approximately 4 000 U/ml. From 5 000 to 250 000 U, preferably approximately 10 000 U to 50 000 U, of oxidoreductase per kg of compound of the formula I to be converted are used. The enzyme unit 1 U in this connection corresponds to the amount of enzyme required in order to convert 1 mmol of ethyl 2-oxo-phenylbutyrate to ethyl S-2-hydroxy-4-phenylbutyrate per minute (min).

The invention further relates to a method for enantioselectively obtaining S-2-hydroxy acid ester, which comprises
a) reducing 2-oxo acid ester to the corresponding S-2-hydroxy acid ester in the presence of oxidoreductase, NADPH and water,
b) reducing at the same time the NADP produced by said oxidoreductase to NADPH with a dehydrogenase and a cosubstrate, and
c) isolating the chiral S-2-hydroxy acid ester produced.

Examples of suitable dehydrogenases are alcohol dehydrogenases from Thermoanaerobium brockii, Lactobacillus kefir or Lactobacillus brevis, said enzymes requiring the coenzyme NADPH (DE 19 610 984, EP 0 456 107, WO 97/32012). Suitable cosubstrates of the alcohol dehydrogenase used are alcohols such as ethanol, 2-propanol (isopropanol), 2-butanol, 2-pentanol or 2-octanol.

The reduction of NADP may also be carried out using the known enzymes used for regenerating NADPH, for example glucose dehydrogenase or NADPH-dependent formate dehydrogenase (Tishkov et al., J. Biotechnol. Bioeng. [1999] 64, 187-193, Pilot-scale production and isolation of recombinant NAD and NADP specific formate dehydrogenase).

The cosubstrate suitable for the method of the invention when using glucose dehydrogenase is glucose. Examples of suitable cosubstrates of formate dehydrogenase are salts of formic acid such as ammonium formate, sodium formate or calcium formate.

Preference is given to using Lactobacillus minor alcohol dehydrogenase (DE 101 19274). It is possible to use in the method of the invention either completely or partially purified alcohol dehydrogenase or whole cells containing said alcohol dehydrogenase. The cells used in this connection may be in native, permeabilized or lysed form. From 10 000 U to 200 000 U, preferably approximately 25 000 U to 100 000 U, of alcohol dehydrogenase are used per kg of compound of the formula I to be converted. The enzyme unit 1 U in this connection corresponds to the amount of enzyme required in order to convert 1 μmol of the cosubstrate (e.g. 2-propanol) per minute (min).

Preference is given to adding to the water a buffer, for example potassium phosphate buffer, Tris/HCl buffer or triethanolamine buffer, having a pH of from 5 to 10, preferably from 6 to 9. The buffer concentration is from 10 mM to 150 mM, preferably from 90 mM to 110 mM, in particular 100 mM. Additionally, the buffer may also contain ions for stabilizing or activating both enzymes, for example magnesium ions for stabilizing Lactobacillus minor alcohol dehydrogenase.

The temperature in the methods of the invention is, for example, from approximately 10° C. to 60° C., preferably from 30° C. to 55° C.

The invention further relates to a method for enantioselectively obtaining S-2-hydroxy acid ester, which comprises
a) reducing 2-oxo acid ester to the corresponding S-2-hydroxy acid ester in the presence of oxidoreductase, NADPH and water,
b) reducing at the same time the NADP produced by said oxidoreductase to NADPH with a dehydrogenase and a cosubstrate,
c) carrying out the reactions in the presence of an organic solvent, and
d) isolating the chiral S-2-hydroxy acid ester produced.

Examples of preferred organic solvents are diethyl ether, tert-butyl methyl ether, diisopropyl ether, dibutyl ether, butyl acetate, heptane, hexane and cyclohexane.

The reaction mixture comprises an aqueous phase and an organic phase when additional solvents are used. The organic phase is formed by a suitable solvent in which the substrate has been dissolved or by the water-insoluble substrate itself. In this connection, the organic phase is from about 5% to 80%, preferably from 10% to 40%, of the total reaction volume.

In the two-phase system of the invention, comprising a first liquid phase and the organic solvent, water forms the second liquid phase. Where appropriate, a solid or another liquid phase which is produced, for example, by incompletely dissolved oxidoreductase and/or alcohol dehydrogenase or by the compound of the formula I may additionally also be present. However, preference is given to two liquid phases without solid phase. Preference is given to mixing said two liquid phases mechanically, so as to generate large surface areas between the two liquid phases.

The concentration of the cofactor NADPH is from 0.001 mM to 0.1 mM, in particular from 0.005 mM to 0.02 mM, based on the aqueous phase.

Preference is given to using in the method of the invention additionally another stabilizer of alcohol dehydrogenase. Examples of suitable stabilizers are glycerol, sorbitol or dimethyl sulfoxide (DMSO).

The compounds of the formula I are used in the method of the invention in an amount of from 10% to 60%, preferably from 15% to 50%, in particular from 20% to 40%, based on the total volume.

The amount of cosubstrate for regenerating NADP to NADPH, such as isopropanol, is from about 5% to 50%, preferably from 10% to 30%, in particular from 15% to 25%, based on the total volume.

The method of the invention is carried out, for example, in a closed reaction vessel made of glass or metal. For this purpose, the components are individually transferred to said reaction vessel and stirred under an atmosphere of nitrogen or air, for example. The reaction time is from 1 hour to 48 hours, in particular from 2 hours to 24 hours, depending on the substrate and the compound of the formula I used.

Subsequently, the reaction mixture is worked up. For this purpose, the aqueous phase is removed and the organic phase is filtered. Where appropriate, the aqueous phase may be extracted once more and, like the organic phase, worked up further. This is followed by evaporating the solvent from the clear organic phase, where appropriate. This results, for example, in the product ethyl S-2-hydroxy-4-phenylbutyrate which is more than 94% enantiomerically pure and essentially free of the reactant ethyl 2-oxo-4-phenylbutyrate. After distillation of the product, the total yield of the process is from 50% to 95%, based on the amount of reactant used.

The invention will be illustrated by the following examples:

### Example 1

### Screening for Oxidoreductases for Reducing 2-oxo Acid Esters in Strains of the Genus Lactobacillus

Various strains of the genus Lactobacillus were cultured for screening in the following medium (figures in each case in g/l): glucose (20), yeast extract (5), meat extract (10), diammonium hydrogen citrate (2), sodium acetate (5), magnesium sulfate (0.2), manganese sulfate (0.05), dipotassium hydrogen phosphate (2). The medium was sterilized at 121° C. and the strains of the genus Lactobacillus (abbreviated to L. hereinbelow) were cultured without further pH regulation or supply of oxygen.

125 mg of cells were then resuspended with 800 μl of disruption buffer (100 mM triethanolamine (TEA), pH 7.0), mixed with 1 g of glass beads and disrupted in a ball mill (Retsch) at 4° C. for 10 min. The supernatant obtained after 2 minutes (min) of centrifugation at 12 000 revolutions per minute (rpm) was used in activity screening and for determining the enantiomeric excess. The substrates used were ethyl 2-oxopentanoate and ethyl 2-oxo-4-phenylbutyrate.

Activity screening mix:

| | | |
|---|---|---|
| 860 | μl | 0.1 M KH₂PO₄/K₂PO₄ pH = 7.0 1 mM MgCl₂ |
| 20 | μl | NADPH/NADH (10 mM) |
| 20 | μl | lysate |
| 100 | μl | substrate (100 mM) |

The reaction was monitored at 340 nm for 1 min.

ee determination mix:

| | |
|---|---|
| 20 | μl lysate |
| 100 | μl NADH/NADPH (50 mM) |
| 60 | μl substrate (ethyl 2-oxo-4-phenylbutyrate 100 mM) |

The reaction mixes for ee determination were extracted with chloroform after 24 hours (h) and the enantiomeric excess was analyzed by means of GC.

The enantiomeric excess is calculated as follows: ee(%)=((R-alcohol-S-alcohol)/(R-alcohol+S-alcohol))×100.

TABLE 1

| | | Ethyl 2-oxo-4-phenyl- | | | | Ethyl 2-oxo-pentanoate | |
|---|---|---|---|---|---|---|---|
| | | butyrate activity in U/g of | | | | activity in U/g of | |
| DSMZ | | host organism cells | | | | host organism cells | |
| No. | Name | NADH | NADPH | ee | | NADH | NADPH |
| 20011 | L. casei | 0 | 0 | — | | 0 | 0 |
| 20019 | L. curvatus var. | 0 | 0 | — | | 0 | 0 |
| | Curvatus | | | | | | |
| 20184 | L. farciminis | 0 | 0 | — | | 0 | 0 |
| 20243 | L. gasseri | 0 | 0 | — | | 0 | 0 |
| 20249 | L. alimentarius | 0 | 0 | — | | 0 | 0 |
| 20494 | L. sakei | 0 | 0 | — | | 0 | 0 |
| 20555 | L. salivarius var. | 0 | 0 | — | | 0 | 0 |
| | Salivarius | | | | | | |
| 20557 | L. jensenii | 0 | 0 | — | | 0 | |
| 20074 | L. delbrueckii | 0 | 0 | — | | 0 | 0 |
| | var. Delbrueckii | | | | | | |
| 20001 | L. coryniformis | 0 | 0 | — | | 0 | 0 |
| | var. caryniformis | | | | | | |
| 20190 | L. halotolerans | 0 | 0 | — | | 0 | 0 |
| 20016 | L. reuteri | 0 | 12.3 | 94% | S | 0 | 21.1 |
| 20003 | L. bifermentans | 0 | 0 | — | | 0 | 0 |
| | L. kefiri | 0 | 2.5 | 26% | S | 0 | 7.7 |
| 4864 | L. oris | 0 | 0 | — | | 0 | 0 |
| 20515 | L. collinoides | 2.6 | 3.4 | 26% | R | 2.5 | 7.7 |
| 20014 | L. minor | 0 | 0 | — | | 0 | 0 |
| 20593 | L. kandleri | 3.6 | 3.6 | 32% | S | 4.3 | 12.8 |
| 5705 | L. parabuchneri | 0 | 5.1 | 38% | S | 0 | 11.3 |
| 20349 | L. fructosus | 0 | 0 | — | | 0 | 0 |
| 20055 | L. cellobiosus | 0 | 12.3 | 92% | S | 0 | 13.3 |
| 20015 | L. reuteri | 0 | 12 | 96.6% | S | 0 | 26 |
| 20049 | L. fermentum | 0 | 7.7 | 82% | S | 0 | 6.8 |

| | | | | | | |
|---|---|---|---|---|---|---|
| 20052 | L. fermentum | 0 | 0 | — | 0 | 0 |

DSMZ stands for Deutsche Sammlung für Mikroorganismen und Zellkulturen, Mascheroder Weg lb, 38124 Braunschweig, Germany.

Table 1 reveals that a plurality of species of the genus Lactobacillus have an NADPH-dependent oxidoreductase with ethyl 2-oxo-4-phenylbutyrate or ethyl 2-oxopentanoate as substrate.

Definition of enzyme units: 1 U corresponds to the amount of enzyme required to convert 1 mmol of substrate per min.

### Example 2

### Isolation of an NADPH-Dependent Oxidoreductase from Lactobacillus Reuteri

An NADPH-dependent oxidoreductase was isolated from Lactobacillus reuteri by culturing the organism as described in example 1. After reaching the stationary phase, the cells were harvested and separated from the medium by means of centrifugation. The enzyme was liberated by wet grinding by means of glass beads but this could also have been achieved by other disruption methods. For this purpose, 20 g of L. reuteri were suspended with 80 ml of disruption buffer (100 mM triethanolamine, 1 mM MgCl₂ pH=7.0) and, after addition of 80 ml of glass beads, the cells were disrupted by means of a ball mill (Retsch, 10 min, 24 Hz).

The crude extract obtained after centrifugation was then adjusted to a final concentration of 50% ammonium sulfate by adding 242 mg of (NH₄)₂SO₄ and stirred at 4° C. for 1 h. The pellet was then removed by centrifugation at 12 000 rpm for 10 min and the supernatant obtained was further purified by means of FPLC. The enzyme was purified using hydrophobic interaction chromatography on Butyl Sepharose Fast Flow (Pharmacia) and subsequent gel permeation (Superdex 200 HR, Pharmacia). For this purpose, the supernatant after ammonium sulfate precipitation was applied directly to a Butyl Sepharose FF column equilibrated with 100 mM TEA pH=7.0 and 1 M (NH₄)₂SO₄ and eluted with a descending linear salt gradient. The enzyme was eluted at 0 M (NH₄)₂SO₄. The active fractions were combined and reduced to a suitable volume by means of ultrafiltration (cut-off 10 kDa). The enzymic activity of oxidoreductase is determined in the assay system according to example 1 (activity screening mix) and the amount of protein was determined according to Lowry et al. Journal of Biological Chemistry, 193 (1951): 265-275 or Peterson et al., Analytical Biochemistry, 100 (1979): 201-220). The specific activity is the enzyme activity divided by the amount of protein, with 1 unit (U) corresponding to the conversion of 1 mmol per min.

The crude enzyme preparation was then further purified by means of gel permeation (TEA 100 mM pH=7.0, 0.15 M NaCl, 1 mM MgCl₂) and the molecular weight of the native enzyme was determined at the same time. The molecular weight standards used were catalase (232 kDa), aldolase (158 kDa), albumin (69.8 kDa) and ovalbumin (49.4 kDa).

Table of Purification

TABLE 2

| | | | Total | Specific | |
|---|---|---|---|---|---|
| Purification | Volume | Activity | activity | activity | |
| step | [ml] | [U/ml] | [U] | [U/mg] | Yield |
| Crude extract | 20 | 9.3 | 186 | 0.25 | 100% |
| (NH₄)₂SO₄ | 20 | 5.4 | 109 | 0.83 | 57% |
| precipitation | | | | | |
| Butyl Sepharose | 1 | 20 | 20 | 54 | 10% |
| Gel permeation | 0.1 | 20 | 2 | 120 | 1.1% |

The molecular weight of the protein in the native state is 60 ± 5 kDa, as determined by means of gel permeation.

### Example 3

### Determination of the N-Terminal Sequence and Determination of an Internal Peptide after in-Gel Digest

After gel permeation, the enzyme preparation was fractionated in a 10% strength sodium dodecyl sulfate (SDS) gel and transferred to a polyvinylidene difluoride membrane (PVDF membrane).

The prominent band at approximately 30 to 35 kDa was subjected to N-terminal sequencing by means of Edman degradation (Procise 492 (PE Biosystems). The following N-terminus sequence was obtained:

| | |
|---|---|
| MKNIMIAGAGVLGSQ: | SEQ ID 1 |

The SDS-PAGE band of the same protein was reduced with dithiothreitol, carboxymethylated and digested with endoproteinase Lys-C. The peptides obtained were separated via a 300 μm×150 mm capillary HPLC column (Vydac RP18, LC Packings). 25 fractions were collected manually and tested by means of MALDI MS (Voyager-DE STR (PE Biosystems) for peptides suitable for sequencing. Fraction 12 with MH⁺=1491.8 was sequenced by means of automated Edman degradation and provided the following sequence:

| | |
|---|---|
| SDYERDLHLTDK: | SEQ ID 2 |

### Example 4

### Cloning of the enzyme

4.1 Cloning of a Specific L. reuteri Gene Fragment with the Aid of PCR (Polymerase Chain Reaction)

Chromosomal DNA was extracted from Lactobacillus reuteri cells according to the method described in “Molecular cloning” by Maniatis & Sambrook. The resulting genomic DNA served as template for the direct polymerase chain reaction (PCR) using degenerated primers. Said degenerated 5′ primers were derived from the N-terminal amino acid sequence (Seq. No: 1) and the 3′ primers were derived from the amino acid sequence of an internal peptide (Seq. No: 2), taking into account the universal gene code (Seq. No: 3, 4, 5 and 6). Primer constructs are listed below.

N=A, T, C or G; Y=T or C; R=A or G

The primers were prepared by known methods.

5′-Oligo 3:
ATGAARAAYATYATGATYGCHGGCGC
5′-Oligo 4:
ATGAARAAYATYATGATYGCHGGTGC
3′-Oligo 5:
RTGHARATCMCGTTCRTAATC
3′-Oligo 6:
RTGHARATCMCGTTCRTAGTC

The amplification was carried out in PCR buffer [10 mM Tris-HCl (pH 8.3), 50 mM KCl, 2.5 mM MgCl₂, 1 mM deoxynucleoside triphosphate mix (dNTP) mix, 30 pmol of each primer and 2.5 U of AmpliTaq Gold (Applied Biosystems)]. After activation of the AmpliTaq Gold polymerase (10 min, 94° C.) and the subsequent 35 PCR cycles (94° C., 60 sec; 53° C., 45 sec; 72° C., 60 sec), the reaction was cooled to 4° C. and the entire PCR mixture was applied to a 1% agarose gel for analysis.

4.2 Subcloning of PCR Amplification Product

A specific fragment of the L. reuteri (S)-ADH gene, identified at 200 bp, was ligated, after gel purification (Qiaex agarose extraction kit), into TA-Cloning Vector pCR 2.1 (Invitrogen, Karlsruhe, Germany). After transformation of 2 μl of the ligation mixture into E. coli top 10F′ cells, DNAs of colonies produced were screened for the presence of the pCR2.1 plasmid with integrated 200 bp PCR fragment. For this purpose, a restriction analysis with Eco RI endo-nuclease was carried out. Subsequently, positive clones were sequenced using the following primers:

M13 rev:
5′ CAGGAAACAGCTATGACC 3′
and
M13 uni:
5′ TGTAAAACGACGGCCAGT 3′

with the aid of an ABI DNA sequencer.

Sequence analysis of the 159 bp gene fragment (Seq. No: 7) revealed an open reading frame of 53 amino acids, which also included the sequence fragments of both the N terminus and the internal peptide.

4.3 Cloning of the Full-Length Gene Segment Coding for L. reuteri (S)-ADH

Based on the nucleotide sequence of the 159 bp gene fragment of example 4.2., specific primer pairs were constructed for an inverse polymerase chain reaction (iPCR) with subsequent nesting PCR (Seq. No: 8, 9, 10 and 11). The primers 8 and 9 are complementary to the 3′ end of the gene fragment found and the primers 10 and 11 are complementary to the region close to the 5′ end.

Oligo 8:
ATCCGGCTTTAATGTCAGCGT
Oligo 9:
CGACGGATTAAGGCGCTGAAAAG
Oligo 10:
CTACCTAATACGCCAGCACCAG
Oligo 11:
GCCAGCACCAGCAATCAT

Chromosomal DNA from L. reuteri cells was digested with Eco RI endonuclease and used for religation with T4 ligase. The resulting circular, chromosomal DNA fragments served as template for the iPCR. The following amplification cycles of a polymerase chain reaction were carried out in a PCR buffer (see example 2), 1 mM MgCl₂, containing in each case 30 pmol of primers 8 and 10, 25 ng of the religation product as template and 2.5 U of AmpliTaq Gold DNA polymerase (Applied Biosystems):

| | | | | |
|---|---|---|---|---|
| Cycle 1: | 94° | C., | 10 | min |
| Cycle 2 × 30: | 94° | C., | 1 | min |
| | 57° | C., | 45 | s |
| | 72° | C., | 1 | min |
| Cycle 3: | 72° | C., | 7 | min |
| | 4° | C., | ∞ | |

The PCR signal was then amplified by a nesting PCR. In this reaction, the optimal MgCl₂ concentration was at 2 mM, with 2 μl of the first iPCR as template. A gradient PCR determined a temperature of 57° C. as being optimal for the primer pair 9 and 11. The following amplification cycles with AmpliTaq Gold DNA polymerase were required in order to be able to detect a specific PCR product of 1 200 bp in length:

| | | | | |
|---|---|---|---|---|
| Cycle 1: | 95° | C., | 10 | min |
| Cycle 2 × 40: | 95° | C., | 45 | s |
| | 57° | C., | 1 | min |
| | 72° | C., | 90 | s |
| Cycle 3: | 72° | C., | 7 | min |
| | 4° | C., | ∞ | |

The specific band of 1 200 bp in length was purified via a 1% agarose gel by means of the Qiaex gel extraction kit (Qiagen, Hilden, Germany) and used in a ligation reaction with the TA-PCR cloning vector pCR2.1 (Invitrogen).

After transformation of 2 μl of the ligation mixture into E. coli top 10F′ cells, plasmid DNAs of ampicillin-resistant colonies produced were screened for the presence of the pCR2.1 plasmid with integrated 1 200 bp PCR fragment. For this purpose, a restriction analysis with Eco RI endonuclease was carried out. Subsequently, positive clones were sequenced as described under 4.2, using the primers M13 rev and M13 uni.

The sequence analysis of the 1 241 bp DNA fragment (Seq. No: 12) revealed an open reading frame of 148 amino acids in the 5′-terminal region. The sequence of the first 15 N-terminal amino acids corresponded to the C-terminal amino acid sequence Seq No: 7 of 4.2. The analysis of the C-terminal sequence of the 1 241 bp DNA fragment revealed enclosed regulatory DNA segments to the N-terminal end of the L. reuteri (S)-ADH gene.

Based on the sequence of the 1 241 bp DNA fragment whose N-terminal region (447 bp) represents another section of the L. reuteri oxidoreductase gene, specific primers for another inverse polymerase chain reaction (iPCR) with subsequent nesting PCR were constructed (Seq. No: 13 and 14). The primers 13 and 14 are complementary to the 3′ extension of the gene fragment found.

Oligo 13:
CCAGAGGTGATTGAAGAAGCTAC
Oligo 14:
CCGGGAAATAAAGATGGTT

Chromosomal DNA from L. reuteri cells was digested with Af1 III endonuclease and used for religation with T4 ligase. The resulting circular chromosomal DNA fragments served as template for the iPCR. The following amplification cycles of a polymerase chain reaction were carried out in a PCR buffer (see example 4.1), 1 mM MgCl₂, containing in each case 30 pmol of primers 7a/9a, 25 ng of the religation product as template and 2.5 U of AmpliTaq Gold DNA polymerase (Applied Biosystems):

| | | | | |
|---|---|---|---|---|
| Cycle 1: | 94° | C., | 10 | min |
| Cycle 2 × 30: | 95° | C., | 1 | min |
| | 56° | C., | 45 | s |
| | 72° | C., | 1:45 | min |
| Cycle 3: | 72° | C., | 7 | min |
| | 4° | C., | ∞ | |

The PCR signal was then amplified by a nesting PCR. The optimal MgCl₂ concentration in this reaction was 2 mM, and 2 μl of the first iPCR were used as template for said nesting PCR. The following amplification cycles with AmpliTaq Gold DNA polymerase were required in order to be able to detect a specific PCR product of 950 bp in length:

| | | | | |
|---|---|---|---|---|
| Cycle 1: | 94° | C., | 10 | min |
| Cycle 2 × 40: | 94° | C., | 45 | s |
| | 56° | C., | 1 | min |
| | 72° | C., | 1:45 | min |
| Cycle 3: | 72° | C., | 7 | min |
| | 4° | C., | ∞ | |

A specific band of 950 bp in length was purified by means of the Qiaex gel extraction kit (Qiagen) via a 1% agarose gel and used in a ligation reaction with the TA-PCR cloning vector pCR2.1 (Invitrogen).

After transformation of 2 μl of the ligation mixture into E. coli top 10F′ cells, plasmid DNAs of ampicillin-resistant colonies produced were screened for the presence of the pCR2.1 plasmid with integrated 950 bp PCR fragment. For this purpose, a restriction analysis with Eco RI endonuclease was carried out. Subsequently, positive clones were sequenced as described in 4.2, using the primers M13 rev and M13 uni.

The DNA fragment inserted into the pCR2.1 vector was 822 bp in length and had at the N terminus an open reading frame of 126 amino acids, which ended with a stop codon and a termination loop (Seq. No: 15). The 5′ peptide of 12 amino acids corresponded to the C-terminal end of sequence No: 12. Thus the 822 bp DNA fragment generated by iPCR comprised the C-terminal end of the gene segment coding for an L. reuteri oxidoreductase.

4.4 Synthesis of the Full Gene of an L. Reuteri Oxidoreductase by Means of PCR

Based on the sequences No: 7 and No: 15, specific primers were constructed for subsequent cloning of the full-length gene into a suitable expression system. The 5′ primer was modified with an Nde I recognition sequence and the 3′ primer was modified with a Hind III recognition sequence (Seq. No: 16; Seq. No: 17).

Oligo 16:
GCGGAATTCCATATGAAGAATATCATGATTGCT
Oligo 17:
CCCAAGCTTAATGCTTCAGAAAATCTGG

Genomic DNA of L. reuteri cells served as template for the polymerase chain reaction. The amplification was carried out in a PCR buffer [10 mM Tris-HCl, (pH 8.0); 50 mM KCl; 10 mM MgSO₄; 1 mM dNTP mix; 30 pmol of each primer and 2.5 U of Platinum Pfx DNA polymerase (Invitrogen)] with 300 ng of template and the following temperature cycles:

| | | | | |
|---|---|---|---|---|
| Cycle 1: | 94° | C., | 2 | min |
| Cycle 2 × 30: | 94° | C., | 15 | s |
| | 58° | C., | 30 | s |
| | 68° | C., | 75 | s |
| Cycle 3: | 68° | C., | 7 | min |
| | 4° | C., | ∞ | |

After purification via a 1% agarose gel, the resulting PCR product was digested with Nde I and Hind III and ligated into the pET21a vector (Novogene, Madison, USA) backbone treated with the same endonucleases. After transformation of 2 μl of the ligation mixture into E. coli Top 10 F′ cells, plasmid DNAs of ampicillin-resistant colonies were checked by means of restriction analysis with endonucleases Nde I and Hind III for correct ligation. The expression construct pET21-reut#10 was sequenced. The Lactobacillus reuteri oxidoreductase gene has an open reading frame of 882 bp in total (Seq. No: 19), corresponding to a protein of 294 amino acids (Seq. No: 18).

4.5 Production of the Recombinant (S)-ADH in E. coli

Competent Escherichia coli StarBL21(De3) cells (Invitrogen) were transformed with the pET21-reut#10 expression construct containing the oxidoreductase gene. The strain was cultured in LB medium (1% tryptone, 0.5% yeast extract, 1% NaCl) containing ampicillin (50 μg/ml), until an optical density, measured at 500 nm, of 0.5 was reached. Oxidoreductase expression was induced by addition of isopropyl-thiogalactoside (IPTG) at a final concentration of 1 mM. After induction at 25° C. and 220 rpm for 8 hours, the cells were harvested and frozen at −20° C.

For the following experiments for biochemical characterization, 100 mg of cells were admixed with 600 μl of disruption buffer and 600 μl of glass beads and disrupted by means of a ball mill for 10 min. The lysate obtained was then used in diluted form for the corresponding measurements. Said lysate had an activity of from 2 000 to 4 000 U/ml with ethyl 2-oxo-4-phenylbutyrate.

Thus, the enzyme was expressed with activities of from 10 000 U/g to 30 000 U/g of wet weight E. coli. The enzyme is thus inexpensive and available in large quantities.

### Example 5

### Characterization of the Recombinant L. Reuteri Oxidoreductase

5.1 pH Optimum

Preparation of the following measurement buffers, all of which are 50 mM, contain 1 mM MgCl₂ and have different pH values.

TABLE 3

| pH | Buffer system |
|---|---|
| 4 | Sodium acetate/acetic acid |
| 4.5 | Sodium acetate/acetic acid |
| 5 | Sodium acetate/acetic acid |
| 5.5 | KH₂PO₄/K₂PO₄ |
| 6 | KH₂PO₄/K₂PO₄ |
| 6.5 | KH₂PO₄/K₂PO₄ |
| 7 | KH₂PO₄/K₂PO₄ |
| 7.5 | KH₂PO₄/K₂PO₄ |
| 8 | KH₂PO₄/K₂PO₄ |
| 8.5 | KH₂PO₄/K₂PO₄ |
| 9 | Glycine/NaOH |
| 9.5 | Glycine/NaOH |
| 10 | Glycine/NaOH |
| 11 | Glycine/NaOH |

Enzyme diluted as required (1:20)

Measurement mix (30° C.):

| | | |
|---|---|---|
| 870 | μl | Measurement buffer with varying pH |
| 20 | μl | NADPH 10 mM (8.6 mg/ml H₂O) |
| 10 | μl | Enzyme, diluted |
| 2-3 | min | incubation |
| +100 | μl | Substrate solution (100 mM ethyl 2-oxo-4-phenylbutyrate) |

The pH optimum was determined by determining the enzymic reaction in the particular buffer listed in table 3. A pH optimum of between 6.5 and 7 was determined for the enzyme of the invention. The enzyme has 80% of its maximum activity in the pH range from 4.5 to 8, with said activity then rapidly decreasing at pH values of below 4.0 and above 8.5.

5.2 pH Stability

The dependence of the activity of the enzyme on storage in buffers of different pH was studied for the pH range from 4 to 11. For this purpose, various buffers (50 mM) in the pH range from 4 to 11 were prepared and the enzyme overexpressed in example 4 was diluted therein 1:200 and incubated for 30, 60 and 300 min. All buffers contained 1 mM MgCl₂. 10 μl thereof were then used in the normal activity assay. The starting value here is the measurement value obtained immediately after diluting the enzyme in potassium phosphate buffer 50 mM pH=7.0. Said value corresponded under predefined conditions to a change in extinction of 0.70/min and was set to 100% and all subsequent measurement values were related to this value.

It was found that the recombinant L. reuteri oxidoreductase is stable in the pH range from 4.5 to 8.0 and may be incubated without loss of activity for at least 5 h. 50% and 40% remaining activity were found for pH 4.0 and 9.0, respectively, after 5 h. pH values above 9.5 lead to immediate inactivation of the enzyme.

5.3 Temperature Optimum

The optimal assay temperature was determined by measuring the enzyme activity in the temperature range from 15° C. to 70° C. in the standard measurement mixture. As table 5 indicates, the maximum activity of the enzyme is at 55° C., and rapidly decreases thereafter.

TABLE 4

| Temper- | Activity | Temper- | Activity |
|---|---|---|---|
| ature | in U/ml of | ature | in U/ml of |
| (° C.) | undiluted enzyme | (° C.) | undiluted enzyme |
| 15 | 385 | 45 | 2900 |
| 20 | 745 | 50 | 3200 |
| 25 | 1090 | 55 | 3800 |
| 30 | 1350 | 60 | 617 |
| 35 | 1860 | 65 | 180 |
| 40 | 2500 | 70 | 90 |

5.4 Temperature Stability

The temperature stability for the range from 15° C. to 70° C. was determined in a manner similar to that described under 5.2. For this purpose, in each case a 1:200 dilution of the purified enzyme was incubated at the particular temperature for 60 min and 180 min and then measured at 30° C. using the above assay mixture. Here too, the starting value used was the measurement value which is obtained immediately after diluting the enzyme in potassium phosphate buffer 50 mM pH=7.0 and which was set to 100% here, too.

The enzyme is totally stable in a temperature range from 15° C. to 50° C. and exhibits no loss of activity whatsoever after 3 h of incubation. At 55° C., an enzyme activity is no longer detectable after only 30 min.

5.5 Substrate Spectrum/Enantiomeric Excess

The substrate spectrum of the oxidoreductase of the invention was determined by measuring the enzyme activity with a number of ketones, oxo acids and esters thereof. For this purpose, the standard measurement mixture (example 5.1) was used with different substrates. The activity with ethyl 2-oxo-4-phenylbutyrate was set to 100% and all other substrates were related thereto. The enzyme exhibited no NADP-dependent dehydrogenase activity for ethyl (R)- or (S)-2-hydroxy-4-phenylbutyrate, (R)- or (S)-4-chloro-3-hydroxybutyrate and (D)- or (L)-ethyl lactate.

The ee value was determined by preparing the following reaction mixture for selected substrates.

| | | | |
|---|---|---|---|
| 100 μl | NADPH | (50 | mM) |
| 60 μl | substrate | (100 | mM) |

and 1 to 2 units of oxidoreductase

The reaction mixtures for ee determination were extracted with chloroform after 24 h and the enantiomeric excess of the resulting alcohol was analyzed by means of GC.

TABLE 5

| | Relative | | | Relative | |
|---|---|---|---|---|---|
| | activity | Stereo- | | activity | Stereo- |
| Substrate | % | selectivity | Substrate | % | selectivity |
| Ketones | | | 3-Oxo acid esters | | |
| 1-Phenyl-2-propanone | 3 | nd | Ethyl 4-chloro- | 16 | 56% R |
| | | | acetoacetate | | |
| 2-Chloro-l-(3-chloro- | 0 | nd | Methyl acetoacetate | 0.3 | 78% S |
| phenyl)-ethan-1-one | | | | | |
| Acetophenone | 0 | nd | Ethyl 8-chloro-6- | 190 | 94% R |
| | | | oxooctanoic acid | | |
| Caprylophenone | 0 | nd | Dimethyl-3-oxo-1,8- | 42 | Racemate |
| | | | octanedioic acid | | 50% |
| 2-Octanone | 1 | Racemate | Ethyl 3-oxo-valerate | 1.3 | nd |
| | | 50% | | | |
| 3-Octanone | 4 | nd | | | |
| Acetone | 0 | nd | | | |
| 2-Oxo acid esters | | | 2-Oxo acids | | |
| Ethyl 2-oxovalerate | 190 | nd | 2-Oxovaleric acid | 0 | nd |
| Ethyl 2-oxo-4-phenyl- | 100 | 98% S | 2-Oxo-3-phenyl- | 0.5 | nd |
| butyrate | | | propionic acid | | |
| Ethyl pyruvate | 2 | 99% S | 2-oxobutyric acid | 0 | nd |
| Ethyl phenylglyoxylate | 2 | | | | |
| Oxidation | | | | | |
| R-2-Hydroxy-4-phenyl- | 0 | nd | 2-Propanol | 0 | nd |
| butyrate | | | | | |
| S-2-Hydroxy-4-phenyl- | 0 | nd | Ethyl D-lactate | 0 | nd |
| butyrate | | | | | |
| S-4-Chloro-3-hydroxy- | 0 | nd | Ethyl L-lactate | 0 | nd |
| butyrate | | | | | |
| R-4-Chloro-3-hydroxy- | 0 | nd | | | |
| butyrate | | | | | |

As table 5 indicates, the recombinant Lactobacillus reuteri oxidoreductase reduces in particular 2-oxo acid esters stereoselectively to the corresponding 2-hydroxy acid esters. The corresponding 2-oxo acids were not accepted as substrates, methyl ketones were also hardly reduced at all, 3-oxo acid esters are partly reduced but mostly not stereoselectively.

5.6 Solvent Stability

The enzyme stability when making contact with organic solvents was studied by diluting the L. reuteri oxidoreductase 1:400 (in the case of water-miscible organic solvents) with the solvent mixtures indicated and incubating at room temperature. Subsequently, 10 μl of the enzyme solution were used in the standard assay mixture. Here too, the starting value was set to 100% after dilution in the buffer (potassium phosphate buffer 100 mM, pH=7.0, 1 mM MgCl₂) and all other values were related thereto.

In the case of the water-immiscible organic solvents, dilution was likewise carried out in potassium phosphate buffer, the same volume of organic solvent was added to the mixture and the mixture incubated at room temperature in a thermal mixer at rpm=170. The activity was measured from the aqueous phase.

TABLE 6

| Activity | 8 h | 24 h | Activity | 8 h | 24 h |
|---|---|---|---|---|---|
| Buffer KPP | 72% | 70% | Ethyl | 0 | 0 |
| 100 mM pH = 7 | | | acetate | | |
| 1 mM MgCl₂ | | | | | |
| 5% Isopropanol | 77% | 77% | Butyl | 74% | 12% |
| | | | acetate | | |
| 10% Isopropanol | 86% | 85% | Diethyl | 32% | 20% |
| | | | ether | | |
| 20% Isopropanol | 100% | 100% | MTBE | 90% | 81% |
| 30% Isopropanol | 0% | 0 | Diisopropyl | 94% | 77% |
| | | | ether | | |
| 5% EtOH | 64% | 65% | Chloroform | 6% | 0% |
| 10% EtOH | 68% | 70% | Hexane | 115% | 100% |
| 20% EtOH | 83% | 80% | Heptane | 113% | 100% |
| 30% EtOH | 77% | 75% | Cyclohexane | 113% | 100% |
| 10% DSMO | 80% | 80% | | | |
| 20% DSMO | 79% | 80% | | | |

As table 6 indicates, L. reuteri oxidoreductase is remarkably stable with respect to organic solvents. Furthermore, the enzyme is even stabilized in organic water-miscible and water-immiscible solvents, compared to incubation in pure buffer.

5.7 Determination of Kₘ and vₘₐₓ for NADPH and ethyl 2-oxopentanoate

Kₘ and vₘₐₓ of NADPH and ethyl 2-oxopentanoate were determined by choosing the following reaction mixtures:

A. Varying NADPH/Constant Substrate Concentration
970 μl Ethyl 2-oxopentanoate solution in potassium phosphate buffer pH=7.0
20 μl NADPH (the final concentrations of 200-5 μM)
10 μl Enzyme solution (1:300)

B. Varying Substrate Concentration/Constant NADPH
970 μl Ethyl 2-oxopentanoate solution in potassium phosphate buffer pH=7.0 (concentrations of from 10 to 0.1 mM)
20 μl NADPH (final concentration 0.2 mM)

101 μl Enzyme solution (1:300)

| | Km | | vmax | |
|---|---|---|---|---|
| NADPH | 0.004 ± 0.0018 | mM | 3080 U/ml | 18 500 U/g |
| | | | | E. coli wet weight |
| Ethyl 2-oxo- | 0.18 ± 0.07 | mM | 3080 U/ml | 18 500 U/g |
| pentanoate | | | | E. coli wet weight |

The enzyme solution used was the 1:300 diluted lysate (600 μl) obtained from 0.1 g of recombinant E. coli cells.

5.8 Preparative Conversions of Ethyl 2-oxo-4-Phenylbutyrate

A. Coenzyme Regeneration with Secondary Alcohol Dehydrogenase from Thermoanerobium Brockii

For a preparative approach, a mixture of 4 ml of potassium phosphate buffer 100 mM, pH=7.0, 2 ml of isopropanol, 4 ml of ethyl 2-oxo-4-phenylbutyrate, 0.064 mg of NADP, 1 000 units of L. reuteri oxidoreductase and 10 mg of Thermoanerobium brockii ADH (Fluka, approximately 100 U, based on oxidation of 2-propanol) was incubated at room temperature with constant mixing for 24 h. After 24 h, the substrate ethyl 2-oxo-4-phenylbutyrate had been completely converted to ethyl 2-hydroxy-4-phenylbutyrate. 97% of the product was the corresponding S-alcohol.

B. Coenzyme Regeneration with Secondary Alcohol Dehydrogenase (KRED 1004, Biocatalytics Inc, Pasadena)

For a preparative approach, a mixture of 4 ml of potassium phosphate buffer 100 mM, pH=7.0, 2 ml of isopropanol, 4 ml of ethyl 2-oxo-4-phenylbutyrate, 0.064 mg of NADP, 1 000 units of L. reuteri oxidoreductase and 1 mg of KRED 1004 (Biocatalytics Inc, Pasadena) was incubated at room temperature with constant mixing for 2 h. After 2 h, the substrate ethyl 2-oxo-4-phenylbutyrate had been completely converted to ethyl 2-hydroxy-4-phenylbutyrate. 98% of the product was the corresponding S-alcohol.

1. An oxidoreductase, which reduces 2-oxo acid esters to the corresponding S-2-hydroxy acid esters in the presence of NADPH and water.

2. The oxidoreductase as claimed in claim 1, which is obtainable from Lactobacillus (L.) reuteri, L. kefiri, L. kandleri, L. parabuchneri, L. cellobiosus or L. fermentum.

3. The oxidoreductase as claimed in claim 1, which has the amino acid sequence according to SEQ ID NO: 18.

4. The oxidoreductase as claimed in claim 1, wherein more than 70% of the amino acids therein are identical to the amino acid sequence SEQ ID NO: 18 and which has a specific activity of more than 1 μmol per mg, based on the conversion of ethyl 2-oxo-4-phenylbutyrate to ethyl S-2-hydroxy-4-phenylbutyrate.

5. The oxidoreductase as claimed in claim 4, wherein from 80% to 99.5%, of the amino acids are identical to the amino acid sequence of SEQ ID NO: 18.

6. The oxidoreductase as claimed in claim 1, which has from 1 to 50 amino acids more or from 1 to 50 amino acids fewer than the oxidoreductase having the amino acid sequence SEQ ID NO: 18 and a specific activity of more than 1 μmol per mg, based on the conversion of ethyl 2-oxo-4-phenylbutyrate to ethyl S-2-hydroxy-4-phenylbutyrate.

7. The oxidoreductase as claimed in claim 4, which has from 1 to 25 amino acids more or fewer than occur in the amino acid sequence of SEQ ID NO: 18.

8. The oxidoreductase as claimed in claim 1 which has the amino acid sequence of SEQ ID NO: 18 and has been modified once, twice, three, four or five times by a water-soluble polymer and has a specific activity of more than 1 μmol per mg, based on the conversion of ethyl 2-oxo-4-phenylbutyrate to ethyl S-2-hydroxy-4-phenylbutyrate.

9. The oxidoreductase as claimed in claim 8, wherein the water-soluble polymer is polyethylene glycol.

10. An oxidoreductase fragment, which represents a fragment of the amino acid sequence SEQ ID NO: 18, said fragment having from 5 to 30 amino acids.

11. The fragment as claimed in claim 10, which is a fragment of SEQ ID NO: 18 having an amino acid chain of from 6 to 25 amino acids, the amino acid sequences SEQ ID NO: 1 or SEQ ID NO: 2.

12. A fusion protein, which represents the oxidoreductase having the amino acid sequence SEQ ID NO: 18 or a fragment thereof having from 5 to 30 amino acids and said oxidoreductase or said fragment thereof being linked at the N terminus or carboxy terminus via a peptide bond to another polypeptide.

13. An antibody, which binds specifically to the oxidoreductase according to SEQ ID NO: 18 or to a fragment thereof according to SEQ ID NO: 1 or SEQ ID NO: 2.

14. An isolated nucleic acid sequence, which codes for the oxidoreductases according to SEQ ID NO: 18, SEQ ID NO: 1 or SEQ ID NO: 2.

15. An isolated DNA sequence of the oxidoreductase catalyzing the reduction of 2-oxo acid esters to corresponding S-2-hydroxy acid esters in the presence of NADPH and water as claimed in claim 1, wherein said DNA sequence is selected from the group consisting of
a) a DNA sequence having the nucleotide sequence according to SEQ ID NO: 7, SEQ ID NO: 12, SEQ ID NO: 15 or SEQ ID NO: 19 or the in each case complementary strands,
b) a DNA sequence hybridizing to one or more of the DNA sequences according to a) or to their complementary strands, said hybridization being carried out under stringent conditions, and
c) a DNA sequence encoding, owing to the degeneracy of the genetic code, a protein which is also encoded by one or more of the DNA sequences according to a) or b).

16. An isolated DNA sequence, wherein more than 70% of the nucleic acid bases are identical to the DNA sequence according to SEQ ID NO: 7, SEQ ID NO: 12, SEQ ID NO: 15 or SEQ ID NO: 19 or to the complementary strands thereof and which encodes a protein having a specific activity of more than 1 mmol per mg, based on the conversion of ethyl 2-oxo-4-phenylbutyrate to ethyl S-2-hydroxy-4-phenylbutyrate.

17. The isolated DNA sequence as claimed in claim 16, wherein from 80% to 99.5% of the nucleic acid bases are identical to the DNA sequence according to SEQ ID NO: 7, SEQ ID NO: 12, SEQ ID NO: 15 or SEQ ID NO: 19.

18. An isolated DNA sequence, which represents a nucleic acid sequence having from 10 to 50 nucleic acid bases and having a sequence corresponding to part of a DNA sequence according to SEQ ID NO: 7, SEQ ID NO: 12, SEQ ID NO: 15 or SEQ ID NO: 19 or to the complementary strand thereof.

19. The isolated DNA sequence as claimed in claim 18, which is a nucleic acid sequence having from 15 to 45 nucleic acid bases.

20. A cloning vector, which has one or more of the DNA sequences as claimed in claim 14.

21. An expression vector, which has one or more of the DNA sequences as claimed in claim 14 and is linked in a suitable manner to an expression control sequence.

22. A host cell, which is a bacteria, yeast, insect, plant or mammalian cell and which has been transformed or transfected with an expression vector as claimed in claim 21.

23. A method for enantioselectively obtaining S-2-hydroxy acid ester, which comprises reducing 2-oxo acid esters in the presence of oxidoreductase as claimed in claim 1, NADPH and water to the corresponding S-2-hydroxy acid ester and isolating the S-2-hydroxy acid ester produced.

24. The method as claimed in claim 23, wherein the 2-oxo acid ester used is a compound of the formula I
R2—C(O)—C(O)—O—R1 (I)
in which
R1 is
1. —(C₁-C₂₀)-alkyl where alkyl is straight-chain or branched,
2. —(C₂-C₂₀)-alkenyl where alkenyl is straight-chain or branched and comprises one, two, three or four double bonds, depending on the chain length,
3. —(C₂-C₂₀)-alkynyl where alkynyl is straight-chain or branched and comprises one, two, three or four triple bonds, where appropriate,
4. —(C₆-C₁₄)-aryl,
5. —(C₁-C₈)-alkyl-(C₆-C₁₄)-aryl,
6. —(C₅-C₁₄)-heterocycle which is unsubstituted or mono- to trisubstituted by halogen, hydroxyl, amino or nitro, or
7. —(C₃-C₇)-cycloalkyl, and
R2 is
1. —(C₁-C₂₀)-alkyl where alkyl is straight-chain or branched,
2. —(C₂-C₂₀)-alkenyl where alkenyl is straight-chain or branched and comprises one, two, three or four double bonds, depending on the chain length,
3. —(C₂-C₂₀)-alkynyl where alkynyl is straight-chain or branched and comprises one, two, three or four triple bonds, where appropriate,
4. —(C₆-C₁₄)-aryl,
5. —(C₁-C₈)-alkyl-(C₆-C₁₄)-aryl,
6. —(C₅-C₁₄)-heterocycle which is unsubstituted or mono- to trisubstituted by halogen, hydroxyl, amino or nitro, or
7. —(C₃-C₇)-cycloalkyl, wherein the radicals as defined above under 1. to 7. are unsubstituted or, independently of one another, mono- to trisubstituted by
a) —OH,
b) halogen such as fluorine, chlorine, bromine or iodine,
c) —NO₂,
d) —C(O)—O—(C₁-C₂₀)-alkyl where alkyl is linear or branched and unsubstituted or mono- to trisubstituted by halogen, hydroxyl, amino or nitro, or
e) —(C₅-C₁₄)-heterocycle which is unsubstituted or mono- to trisubstituted by halogen, hydroxyl, amino or nitro.

25. A method for enantioselectively obtaining S-2-hydroxy acid ester, which comprises
a) reducing 2-oxo acid ester to the corresponding S-2-hydroxy acid ester in the presence of oxidoreductase as claimed in claim 1 NADPH and water,
b) reducing at the same time the NADP produced by said oxidoreductase to NADPH with a dehydrogenase and a cosubstrate, and
c) isolating the chiral S-2-hydroxy acid ester produced.

26. The method as claimed in claim 25, wherein the 2-oxo acid ester used is a compound of the formula I as claimed in claim 24.

27. The method as claimed in claim 25, wherein the dehydrogenase used is the alcohol dehydrogenase from Thermoanaerobium brockii, Lactobacillus kefir or Lactobacillus brevis and the cosubstrates used are ethanol, 2-propanol, 2-butanol, 2-pentanol or 2-octanol.

28. The method as claimed in claim 25, wherein the dehydrogenase used is glucose dehydrogenase and the cosubstrate used is glucose or the dehydrogenase used is NADPH-dependent formate dehydrogenase and the cosubstrate used is a salt of formic acid, such as ammonium formate, sodium formate or calcium formate.

29. A method for enantioselectively obtaining S-2-hydroxy acid ester, which comprises
a) reducing 2-oxo acid ester to the corresponding S-2-hydroxy acid ester in the presence of oxidoreductase as claimed in claim 1, NADPH and water,
b) reducing at the same time the NADP produced by said oxidoreductase to NADPH with a dehydrogenase and a cosubstrate,
c) carrying out the reactions in the presence of an organic solvent, and
d) isolating the chiral S-2-hydroxy acid ester produced.

30. The method as claimed in claim 29, wherein the 2-oxo acid ester used is a compound of the formula I as claimed in claim 24.

31. The method as claimed in claim 29, wherein the dehydrogenase used is the alcohol dehydrogenase from Thermoanaerobium brockii, Lactobacillus kefir or Lactobacillus brevis and the cosubstrates used are ethanol, 2-propanol, 2-butanol, 2-pentanol or 2-octanol.

32. The method as claimed in claim 29, wherein the dehydrogenase used is glucose dehydrogenase and the cosubstrate used is glucose or the dehydrogenase used is NADPH-dependent formate dehydrogenase and the cosubstrate used is a salt of formic acid, such as ammonium formate, sodium formate or calcium formate.

33. The method as claimed in claim 29, wherein the organic solvents used are diethyl ether, tert-butyl methyl ether, diisopropyl ether, dibutyl ether, butyl acetate, heptane, hexane or cyclohexane.

34. The method as claimed in claim 29, wherein the organic phase is from 5% to 80%, of the total reaction volume.
